(19) United States
(12) Patent Application Publication
(10) Pub. No.: US 2003/0138847 A1
(43) Pub. Date: Jul. 24, 2003

(54) Dosing form for a polymer support, use of said dosing form in organic chemical synthesis and method for production of said dosing form

(75) Inventors: Thomas Ruhland, Valby (DK); Per Holm, Vanlose (DK); Kirsten Schultz, Roskilde (DK); Jannie Egeskov Holm, Valby (DK); Kim Andersen, Virum (DK)

Correspondence Address: DARBY & DARBY P.C., 805 Third Avenue, New York, NY, 10022, US

(73) Assignee: H. Lundbeck A/S, Valby-Copenhagen (DK)

(21) Appl. No.: 10/245,839

(22) Filed: Sep. 16, 2002

Related U.S. Application Data
(63) Continuation of application No. PCT/DK01/00184, filed on Mar. 16, 2001.

(30) Foreign Application Priority Data
Mar. 17, 2000 (DK) PA 2000 00450

(51) Int. Cl. G01N 33/53; C08G 63/91; G01N 33/543; C08G 63/48

(52) U.S. Cl. 435/7.1; 436/518; 525/54.5; 530/333

(57) ABSTRACT

A dosing form for a polymer support for organic chemical synthesis in a solvent medium comprising a fixed weight amount of beads of a polymer containing functional groups, which polymer is insoluble in the solvent for the intended synthesis, is provided as compressed tablets of essentially equal weight and composition wherein the polymer beads are essentially intact and are released as such when the tablets are disintegrated in said solvent.

Use of the dosing form is made in conventional synthesis, in parallel synthesis, in split and mix synthesis and/or combinatorial chemistry.

In a method for producing the dosing form, beads of a polymer having functional gropusgroups is compressed into tablets after pre-treatment with an aprotic organic solvent groups are compressed into tablets after pre-treatment with an aprotic organic solvent.

This application is a continuation of International Application No. PCT/DK01/00184, filed Mar. 16, 2001. The prior application is hereby incorporated by reference, in its entirety.

### FIELD OF THE INVENTION

The present invention relates to the dosing of solid supports in the field of organic chemical synthesis. In particular the invention deals with such dosing forms for use in parallel synthesis or mix and split synthesis in the organic chemical field e.g. combinatorial chemistry and medicinal chemistry.

### BACKGROUND FOR THE INVENTION

Parallel synthesis and split and mix synthesis have become important tools in the search for new compounds in e.g. the pharmaceutical industry. Using these concepts, large numbers of compounds are synthesised. Parallel synthesis is a particular form for organisation of chemical syntheses where a large number of chemical syntheses simultaneously are performed separately in order to obtain a large number of new single compounds, typically for research purposes. For example parallel synthesis can be used to generate a large number, often hundreds or more, of analogues of a particular molecule in order to determine which analogues have the most desirable activities in a specific assay. Split and mix synthesis is another form for organisation of organic synthesis where a large number of compounds are synthesised as mixtures of compounds.

Combinatorial chemistry is a form of parallel synthesis and split and mix synthesis where the order and the features of the individual steps are selected using a particular combinatorial approach.

In order to carry out parallel syntheses, a large number of additions and separations of substances are necessary. In the syntheses relevant in connection with the present invention, functionalized polymers in the shape of beads, i.e. particles or small bodies, serve as solid supports on which desired products are built up. The polymers may alternatively serve to bind non-desired materials in the synthesis medium, i.e. to perform a so-called scavenger action, or to bind synthesis products, i.e. to perform a so-called resin capture action, or to bind catalysts or reagents. After the polymers have served as solid supports they have to be separated from the synthesis medium containing e.g. organic solvents, non-reacted reagents and/or products, and it is desired that this can be done by filtration.

When using a solid support, it is important that the support is stable so that the beads forming the support are not degraded to smaller particles or transformed in other respects which would reduce the filterability and thus the advantage of easy separation by filtration.

In certain parallel syntheses where a large number of reactions are performed simultaneously, the time consumed by the individual weighing out and distribution of the required functionalized polymer substances is considerable. Further errors and mistakes inevitably occur during the required large number of individual weighing.

Also in split and mix synthesis, a considerable number of individual reactions including weighings and distributions have to be performed and, consequently, errors and mistakes are introduced into the synthesis.

Additionally, the support substances may be hygroscopic and oxygen sensitive and thus require special measures which are time consuming and may confer additionally inaccuracy e.g. due to partially reduced functionality of the polymer.

Further, contact with the polymer substances especially during weighing may involve a health risk to the staff performing the syntheses.

Thus there is a need for simple dosing means as alternative to the weighing out and distribution of polymers hitherto used in parallel synthesis and split and mix synthesis in order to reduce the time consumption and increase the through-put of the synthesis, decrease the health risk to the personnel, and protect the functionality of the polymer against the deteriorating effect of oxygen and moisture.

Pre-weighed resin capsules for parallel synthesis have been marketed by Argonaut Technologies Inc. (San Carlos, Ca, US) where the pre-weighed resin is contained in a capsule which is readily soluble in a wide range of organic solvents. WO 99/04895 discloses dosing forms for solid support polymers comprising capsules as well as pouches and coated tablets wherein the core of said coated tablets contains a 1:1 mixture of the polymer support and polyethylene glycol. However, the dissolved capsule, pouch or tablet excipients thus add material to the liquid phase which often is not desirable, and said added material may need to be removed through a washing step.

Atrash et al. (Atrash, B. et al. Angew. Chem. Int. Ed. 2001, 40, No. 5) discloses tablets where the polymer beads are entrapped in an inert polymer matrix which does not disintegrate when suspended in organic solvents.

It should be observed that the use of tablets as dosing form for different types of substances is conventional within other technical areas. Thus, in the pharmaceutical industries, drugs for oral administration are compressed into tablets, usually together with various extenders and adjuvants. These tablets as well as tablets produced in other industries such as detergent tablets are intended for disintegration and at least partial dissolution in aqueous environment, and not disintegration without dissolution in the solvent in order to obtain a filterable dispersion without adding soluble materials to the soluble phase. Further, the problem of recovering particulate material formed by use and disintegration of the tablets by filtration does not exist.

It has now been found that the above mentioned problems can be solved by a new and inventive process for the manufacture of a dosing form wherein the polymer support is comprised in tablets with the amount and type of tabletting excipients allowing that the tablets can be used for direct dosage in synthetic and analytical chemistry such as parallel synthesis, split and mix synthesis or combinatorial chemistry without any washing step. When introduced in the synthesis medium, the tablets disintegrate and release the polymer support beads which regain their shape.

### SUMMARY OF THE INVENTION

Thus the invention deals with a dosing form for a polymer support for organic chemical synthesis. This dosing form comprises a fixed weight amount of beads of a polymer containing functional groups, characterized in being compressed tablets of essentially equal weight and composition wherein the polymer support is essentially intact and is released as such when the tablets are disintegrated in said solvent.

In particular the invention relates to dosing forms for use in organic chemical synthesis where many separated syntheses are performed such as in parallel synthesis, mix and split synthesis and combinatorial chemistry.

The dosing form according to the invention provides a support for solid phase synthesis or can act as scavengers to remove a certain compound from a liquid to which it is added or to capture products from a reaction medium.

In a preferred embodiment, the dosing form further comprises a polymer without functional groups. This auxiliary polymer may be provided to influence the characteristics of the formed tablets and/or to facilitate the tablet pressing.

Further the invention provides a method for production of tablets using conventional tabletting equipment.

In a further preferred embodiment, a pre-treatment of the polymer before tablet compression is provided to improve the flowability, compressibility and dosing of the polymer and therefore reducing the variation in weight and crushing strength of the tablets.

The tablets can be formed using conventional tabletting equipment without damaging the polymer beads in such a way that the filterability of the resulting dispersion is affected.

In still a further preferred embodiment, an addition of a disintegrant such as dimethylated polyethylene glycol (e.g. DM-PEG 2000) increases the ability of the tablet to disintegrate and disperse in a particular solvent.

It is a particular feature of the invention that the formed tablets are able to disintegrate in a particular solvent to provide a dispersion of the beads in such a way that the beads are suitable for the intended reaction and that the dispersion readily can be separated by filtration.

The polymer beads for use in dosing forms according to this invention may be of any polymer capable of performing the desired function as support and which is insoluble in the relevant solvents, capable of being compressed into tablets with or without suitable adjuvants, capable of disintegrating in said relevant solvents when compressed into a tablet and able to reshape as beads after the disintegration of the tablet.

Preferred polymers in the dosing forms according to the invention are functionalized polystyrene based resins such as polystyrene cross linked with divinyl benzene (DVB), including polyethylene glycol grafted resins such as the Tentagel® and Argogel® resins, linear polystyrene, polystyrene resins cross linked with polyethylene glycol including the POEPS (Renil and Meldal, Tetrahedron Letters 37, 6185-88, 1996), and POEPS-3 resins (Buchardt and Meldal, Tetrahedron Letters 39, 8695-8698, 1998), polystyrene resins cross linked with polyoxybutylene such as the poly(styrene-tetrahydrofuran) resins (JandaGel®) (Toy, P. M.; Janda U. D. Tetrahedron. Lett. 1999, 40, 6329-32), polyoxyethylene polyoxy propylene (POEPOP) resins (Renil and Meldal, supra).

In a further preferred embodiment, the polymers are co-polymerised together with additives to achieve special properties of the beads such as magnetic properties by addition of magnetites or magnetites captured in highly cross linked polystyrene particles (Scholeiki, I., Perez, J. M. Tetrahedron Lett. 1999, 40:3531-3534 and Prof Mark Bradley, Dep. of Chemistry, University of Southampton, Presentation at the Conference “High-throughput Synthesis”, Feb. 9-11, 2000).

Generally polymers are marketed as a particulate material where the particles may have different shapes and forms depending on the manufacturing of the polymer. According to the present invention, the polymer is used in form of beads which means small bodies, particles or pellets, where the surfaces are essential smooth and convex and the longest dimension is not larger than 3 fold of the shortest dimension. The forms of the beads may for example be spherical, drop-shaped and ellipsoid.

The size of the polymer particles as beads used according to the invention is selected to enable good filterability, which is promoted by large particles, balanced with a desire for a reasonably high specific surface area, which is promoted by small particles. The particle size of the polymer beads is according to the invention preferably selected in the range 20-600 mesh, more preferably 100-400 mesh.

Functional groups attached to the polymer according to the invention are groups that can take part in a desired reaction to perform an intended effect. Thus functional groups can have the function of binding a soluble compound to the solid phase in order to ease the separation of said compound from the solution. A polymer containing functional groups can, as mentioned, be used as a support in a solid phase synthesis or it can be used as a scavenger to remove undesired compounds from the solution or it can bind catalysts and reagents. A functional group can serve as a reagent or catalyst in a solution phase reaction. The functional groups on a polymer may all be the same groups or they may consist of two or more species.

Polymers containing functional groups including resin bound reagents, reactive groups and catalysis are presently available on the market. These polymers may be used in this invention and include: the TentaGel® resins (Rapp polymere GmbH, Tübingen, Germany), ArgoGel® resins (Sigma-Aldrich), Merrifield-resin and Novasyn® resins (Calbiochem-Novabiochem AG Schwizerland), in particular: TentaGel S AC, TentaGel S Trt, TentaGel S PHB, TentaGel S HMB, TentaGel S AM, TentaGel S RAM, ArgoGel™AS—SO₂NH₂, ArgoGel™-Cl, ArgoGel™-MB-CHO, ArgoGel™-MB-OH, ArgoGel™-NH₂, ArgoGel™-OH, ArgoGel™-Rink, ArgoGel™-Wang, ArgoGel™-Wang-Cl, aminomethylated poly(styrene-co-divinylbenzene), polymer bound piperidine, polymer bound 4-benzyloxybenzaldehyde, polymer bound isocyanate, polymer bound diethylenetriamine, vinylsulfonylmethyl polystyrene, acryloyl Wang resin, chloromethylpolystyrene-divinylbenzene, brominated Wang resin, 2-chlorotrityl chloride resin, brominated PPOA resin, NovaSyn® TGT alcohol resin, trityl chloride resin, 4-methyltrityl chloride resin, 4-methoxytrityl chloride resin, NovaSyn® trityl alcohol resin, NovaSyn® TG bromo resin, NovaSyn® dichlorotrityl alcohol TG resin, bromo-(2-chlorophenyl)methyl polystyrene, bromo-(4-methoxypbenyl)methyl polystyrene, 4-bromo polystyrene, 4-(bromomethyl) phenoxyethyl polystyrene, bromoacetamidomethyl NovaGel™, bromomethylphenyl-acetamidomethyl NovaGel™, p-nitrophenyl carbonate Wang resin, p-nitrophenyl carbonate Merrifield resin, formylpolystyrene, NovaSyn® TG acetal resin, 2-(4-formyl-3-methoxyphenoxy)ethyl polystyrene, 2-(3,5-dimethoxy-4-formylphenoxy)ethoxymethyl polystyrene, 4-(4-formyl-3-methoxyphenoxy)butyryl NovaGel™ HL, carboxypolystyrene HL, 4-methylbenzhydrylamine resin LL, aminomethylated polystyrene HL, 4-(2′,4′-dimethoxyphenyl-FMOC-aminomethyl)-phenoxyacetamido-norleucin-MBHA resin, 4-methylbenzhydrylamine resin HL, hydrazine 2-chlorotrityl resin, 9-FMOC-amino-xanthen-3-yloxy-methyl resin, NovaSyn® TG amino resin HL, 4-sulfamylbutyryl AM resin, 4-sulfamylbenzoyl AM resin, 4-sulfamylbenzoyl NovaSyn® TG resin, amino-(4-methoxyphenyl)methyl polystyrene, aminomethyl NovaGel™, Rink amide NovaGel™, 4-sulfamylbenzoyl NovaGel™, 4-FMOC-hydrazinobenzoyl AM resin, N-benzylaminomethyl polystyrene, piperazinomethyl polystyrene, N-methylaminomethyl polystyrene, Wang resin, Rink acid resin, oxime resin LL, HMPB-BHA resin, 4-Hydroxymethylphenoxyacetyl PEGA resin, hydroxymethyl polystyrene, 4-hydroxymethylbenzoic acid PEGA resin, 4-hydroxymethylbenzoic acid AM resin, 4-(2′,4′-dimethoxyphenyl-hydroxymethyl)-phenoxy resin, methylisocyanate polystyrene HL, tris-(2-aminoethyl)-amine polystyrene HL, morpholinomethyl polystyrene HL, N-(2-aminoethyl)aminomethyl polystyrene, bis-HOBt-ethylenediamine methyl polystyrene, cysteamine methyl polystyrene, thiocarbamoyl AM resin, N-cyclohexylcarbodiimide, N+-methyl polystyrene HL, 4-(N-benzyl-N-methylamino)-pyridine polymer supported, ethylene glycol polymer bound, 4-maleimidobutyramidomethyl-polystyrene, polyethylene glycol 600 bound to polystyrene-1% DVB, poly(4-vinylpyridine), poly(4-vinylpyridinium hydrochloride), poly(4-vinylpyridinium dichromate), poly[4-vinylpyridinium poly(hydrogen flouride)], poly(4-vinylpyridinium p-toluenesulphonate), poly(4-vinylpyridinium tribromide), pyridinium chlorochromate polymer bound, pyridinium toluene-4-sulfonate polymer bound, sulfur trioxide pyridine complex polymer bound, thiocyanate polymer supported, 1,5,7-triazabicyclo[4.4.0]dec-5-ene on polystyrene, tributylmethylammonium chloride polymer bound, tributylmethylphosphonium chloride polymer bound and triphenylphosphine polymer bound.

Further polymers containing functional groups are listed by Ley et al. (Ley, S. V. et al.; J. Chem. Soc., Perkin Trans. 1, 2000, 3815-4195)

Further polymers containing functional groups may be produced using methods well known within the area, and in particular the commercially available polymers may be modified, e.g. by substitution, using techniques well-known for the skilled person.

Further polymers containing functional groups may be derived by the linking of starting material for parallel synthesis by methods well-known for the chemist skilled in the art.

In one embodiment, the dosing form also comprises polymer beads without functional groups. Such a polymer may be used in the dosing form in order to obtain tablets with more desired properties. Generally the polymers without functional groups should be insoluble and inert when present in the dispersion after disintegration. The polymer without functional groups should also be selected to secure good filterability. The size of the particles of polymer without functional groups should be in the same range as for the polymer having functional groups.

The beads of polymer having functional groups and the beads of polymer without functional groups may for a given tablet composition be selected to have similar forms and dimensions or to have different forms and dimensions.

The polymer without functional groups may be used as a filler, which can be used to achieve a more desired tablet size or tablet property than would have been obtained using only the polymer containing functional groups; as tablet matrix or binder; as a stabilizer, which may be used to improve the mechanical stability of the tablet; or as a disintegrating agent, which may be used to improve the disintegration properties of the tablets. Some polymers may even possess properties that make them usable for more than one of the above mentioned functions.

Other additives known within the tabletting area may be used provided that they are chemically inert and insoluble or otherwise acceptable in the reaction medium for which the tablets are intended, for example may silicon(IV)oxide be added, e.g. to avoid problems caused by static electricity.

Polymers used as a stabilizer can be employed in cases where the polymer containing functional groups alone would provide tablets with unacceptable mechanical stability. Examples of polymers that may be used as stabilizers include polystyrenes and alkylated polyglycols.

Polymers used as a disintegration agent may be used to improve the disintegration of the tablets, or to reduce the necessary disintegration time in a chosen solvent. A disintegration agent may for example be used to obtain disintegration in strongly polar, aprotic organic solvents such as acetonitrile or protic organic solvents such as methanol or ethanol. A preferred disintegrating agent is dimethylated polyethylene glycol (DM-PEG), preferably DM-PEG with a molecular weight around 2000 Da (DM-PEG 2000) or higher. While tablets made of polystyrene disintegrate well in methylene chloride, i.e. an moderately polar aprotic solvent, but not in a strongly polar aprotic solvent such as acetonitrile or a protic solvent such as ethanol, tablets containing a mixture of polystyrene and DM-PEG 2000 in a ratio of e.g. 9:1 disintegrate in both methylene chloride, acetonitrile and ethanol within a reasonable time. Preferably the amount of polyethylene glycol (PEG) does not exceed 20%, more preferred it does not exceed 10%, suitably the amount of PEG is zero. Preferably the amount of other tabletting additives as well does not exceed 20%, more preferred it does not exceed 10%, suitably the amount of tabletting additives is zero.

Other additives known within the tabletting area may be used provided that they are chemically inert and insoluble or otherwise acceptable in the reaction medium for which the tablets are intended.

The formation of the tablets may be performed in an inert atmosphere in order to prevent deterioration of the polymer due to oxidation by oxygen or absorption of moisture from the atmosphere. As an inert atmosphere, any inert gas may be used as it will be known within the area. Examples of suitable gases for the inert atmosphere are nitrogen and argon.

Tablet formation can be done using conventional tabletting techniques. The polymer or the mixture containing said polymer is formed into tablets by application of a certain mechanical force, possibly after granulation, using a tabletting machine as it will be known within the art.

Tablets may be formed containing various amounts of the polymer support for example in amounts in the range of 5-5000 mg. The tablets may be compressed to a desired form and size for example to fit in a device such as a tablet dispenser.

The tablets must have a sufficiently high stability to avoid breaking during package, transportation and dispensing. The crushing strength is a measure for the mechanical stability of tablets. The crushing strength of the tablets must be higher than 5 N preferably higher than 10 N, in order to have a satisfactory mechanical stability.

It has turned out that a pre-treatment of the polymer may have a strongly beneficial effect on the quality of the tablets containing said polymer and, in some cases, even be an essential pre-requisite for tablet formation. The pre-treatment is made by suspending the polymer in an aprotic organic solvent. The polymer is filtered off and dried whereafter it is ready for tablet formation. For some polystyrene based polymers, the quality of the obtained tablets in respect of the crushing strength is significantly improved by such pre-treatment of the polymer. Preferred solvents for use in the pre-treatment are methylene chloride and tetrahydrofuran.

One possible explanation, which should not be construed as limiting the scope of the patent, of the effect of treating the polymer support or mixture of polymer support and additives, is that the surface of the polymer beads are partially swollen by the aprotic organic solvent resulting in agglomeration of the polymer beads with the effect that the treated polymer powder has better flowability and may be compressed into tablets with improved mechanical properties.

The dosing form according to this invention may be composed to be used in any protic or aprotic solvent that is suitable for the intended synthesis. In this context, the term “suitable” means that the solvent is capable of dissolving the reagents of the reaction as desired, and do not take part in unintended reactions with other components of the reaction mixture under the condition applied. The solvent may even be a reagent in the intended reaction for instance if methanol is the solvent in a methoxylation reaction.

That a tablet is capable of disintegration in a solvent means that the tablet with application of a minimal mechanical force such as by vortex mixing can disintegrate in the solvent within 30 minutes, preferably within 10 minutes, more preferred within 5 minutes to form a uniform dispersion.

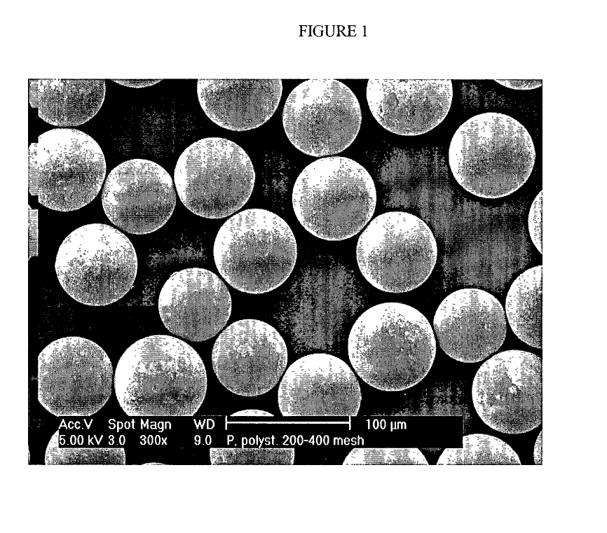

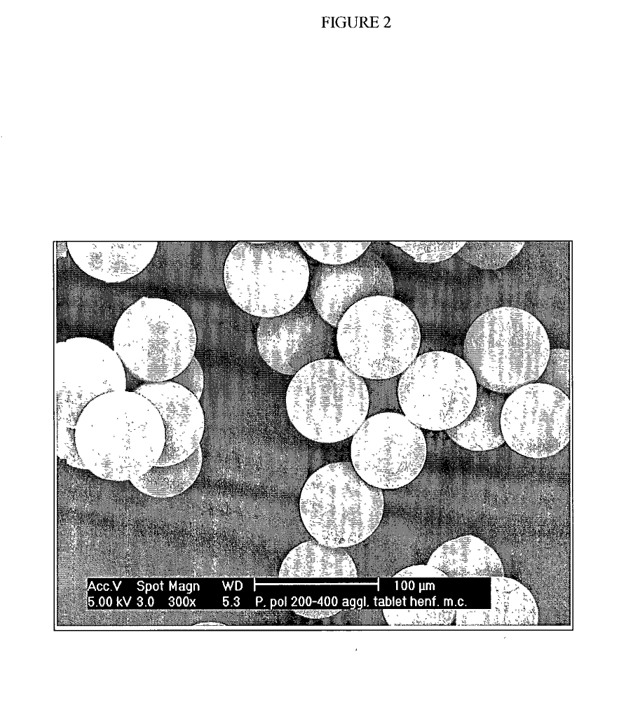

The term “capable of reshaping after the disintegration” means that the polymer beads regain essentially their original shape after the disintegration of a tablet comprising said beads. Further it means that the beads are not mechanical damaged by the tablet compression and subsequent disintegration. Reshaping of the beads can conveniently be evaluated by comparison of scanning electron microscopy (SEM) pictures of the beads before tablet formation and after disintegration. If the beads are capable of reshaping, the shapes of the beads are not substantially altered and the number of cracks and faults in the beads after the dispersion is not substantially higher than before the tablet formation, cf. FIG. 1 and FIG. 2 for further details.

### BRIEF DESCRIPTION OF FIG. 1 AND FIG. 2

FIG. 1: SEM of polystyrene beads, 200-400 mesh, before tablet compression.

FIG. 2: SEM of the polystyrene beads after disintegration of the tablets in methylene chloride.

For Scanning Electron Microscope (SEM) pictures, the samples were sputter coated with gold/palladium and SEM analysis was performed using a Philips electron microscope XL30.

In FIG. 1, the polystyrene beads can be seen as separate uniform spherical bodies in a narrow range of sizes.

In FIG. 2, polystyrene beads as uniform spherical bodies can be seen. The observed beads are all intact without noticeable cracks or damages.

The dosing forms according to the invention are stable and reliable dosing forms that can easily, safely and reliable be distributed to a large number of individual reactions in any form of parallel synthesis and thus increase the throughput of parallel synthesis in a reliable and accurate way.

The invention is now illustrated by specific examples which are presented for illustratory purposes alone and should not be construed as any limitation of the invention.

### EXAMPLES

General procedures

All reactions were carried out under positive pressure of nitrogen. Unless otherwise noted, starting materials were obtained from commercial suppliers and used without further purification. Tetrahydrofuran (THF) was distilled under N₂ from sodium/benzophenone immediately prior to use. DMF was dried over molecular sieve (4 Å) prior to use. Parallel reactions were carried out in a microblock with 96 reactors (1 mL) equipped with polyethylene frits from MultiSynTech (Witten, Germany). The reactors were flushed with nitrogen prior to the reaction. For solid-phase extraction, Scharlau 60 (230-400 mesh) silica gel (sorbil) was used. Ion-exchange chromatography was performed on a Gilson ASPEC XL instrument using SCX-columns (1 g) from Varian Mega Elut®, Chrompack (cat. No. 220508). Prior to use, the SCX-columns were pre-conditioned with 10% solution of acetic acid in methanol (3 mL). LC-MS data were obtained on a PE Sciex API150EX equipped with a Heated Nebulizer source operating at 425° C. The LC pumps were Shimadzu 8A series running with a Waters C-18 4.6×50 mm, 3.5 μm column. Solvent A 100% water+0.05% trifluoroacetic acid, solvent B 95% acetonitrile 5% water+0.035% trifluoroacetic acid. Gradient (2 ml/min): 10% B-100% B in 4 min, 10% B for 1 min. Total time including equilibration, 5 min. Injection volume 10 μL from a Gilson 215 Liquid Handler. The compression of tablets was performed at a Korsch EK0 single punch machine. The crushing strength was measured at a Schleuniger 6D tablet hardness tester. Disintegration times of tablets were measured in a glass tube (16×100 mm) with 2 mL solvent by vortex mixing at a speed of approximately 500 Hz with an IKA shaker (KS 125 basic). The process of tablet disintegration was monitored visually and tablets were deemed to be fully disintegrated when a dispersion was formed and no more lumps were present. For Scanning Electron Microscope (SEM) pictures, the resin samples were sputter coated in a Microtech, Polaron SC 7640 using a gold/palladium electrode and SEM analysis was performed using a Philips electron microscope XL30.

Polystyrene resin was purchased from Rapp Polymere GmbH (Tübingen, Germany) (cat.-No H 1000, 100-200 mesh, cross-linked with 1% divinylbenzene). Dimethylated polyethylene glycol (DM-PEG; molecular weight app. 2000 Da) was purchased from Clariant GmbH (Gendorf, Germany) (Material was grinded in a laboratory blender and sieved, particle size varies). Aminomethyl polystyrene was purchased from Rapp Polymere GmbH (Tübingen, Germany), (cat.-No H 400 02, 1.0 mmol/g; 200-400 mesh; cross-linked with 1% divinylbenzene) Wang-Resin was purchased from Rapp Polymere GmbH (Tübingen, Germany), (cat.-No: H 1011, 1.0 mmol/g; 100-200 mesh; cross-linked with 1% divinylbenzene). Diphenylphosphanyl polystyrene was purchased from Senn Chemicals (Dielsdorf, Switzerland) (cat.-No 40258; 1.69 mmol/g; 100-200 mesh, cross-linked with 1% divinylbenzene). 3-(Morpholino)propyl polystyrene sulphonamide was purchased from Argonaut (USA) (Cat.-No P/N 800282; approximately 2.0 mmol/g). Isocyanato methyl polystyrene (approximately 1 mmol/g; 100-200 mesh and 200-400 mesh; cross-linked with 1% divinylbenzene) was prepared analogously to Booth, R. J.; Hodges, J. C. J. Am. Chem. Soc 1997, 119: 4882-4886, starting from aminomethyl polystyrene. (Vinylcarbonyloxymethyl)phenoxymethyl polystyrene (approximately 0.9 mmol/g; 200-400 mesh; cross-linked with 1% divinylbenzene) was prepared analogously to Morphy, R. J., Rankovic, Rees, D. C. Tetrahedron Lett. 1996, 37: 3209-3212, starting from Wang-Resin. 4-[(4-Nitrophenoxy)carbonyloxymethyl]phenoxymethyl polystyrene (approximately 0.83 mmol/g; 200-400 mesh; cross-linked with 1% divinylbenzene) was prepared analogously to Zaragoza, F.; Tetrahedron Lett. 1995, 47, 8677-8678, starting from aminomethyl polystyrene.

### Example 1

Agglomeration of Functionalised Polystyrene Resin Beads

Wang Resin

Wang-resin beads (25.0 g) was suspended in methylene chloride (150 mL) at room temperature for 15 minutes. The resin was filtered on a D3-frite by gravity and dried on the frite at room temperature in vacuo.

The following agglomerates were prepared according to the procedure described above: 4-[(4-Nitrophenoxy)carbonyloxymethyl]phenoxymethyl polystyrene, 3-(morpholino)-propyl polystyrene sulphonamide, isocyanato methyl polystyrene, (vinylcarbonyloxymethyl)phenoxymethyl polystyrene, diphenylphosphanyl polystyrene.

Tablet Compression

The dried agglomerated material was gently crushed by mortar and pistil and screened through a screen size of 710 μm and transferred to the filling device of the single punch tabletting machine. PEG was mixed with the agglomerated material prior to tabletting if part of the recipe. The tabletting was performed either manually (10-20 tablets) or automatically with a tabletting speed of 50-90 tablets per hour referred to as up-scaling. The compression force was controlled at a value resulting in tablets having a crushing strength of 8-25 N. Tablets with weights in the range 30 mg −200 mg were produced. The punch diameters used were in the range of 48 mm with compound cup shape.

Using the described general procedure, tablets with compositions and crushing strength as indicated in Table 1 were produced.

TABLE 1
Composition of formed tablets

| | | | Tablet | Tablet | Crushing |
|---|---|---|---|---|---|
| Tablet | | Pre-treat- | weight | diameter | strength |
| code | Polymer type | ment | (mg) | (mm) | (N) |
| CP-1 | P1 | A | 100 | 6 | app 8 |
| CP-2 | P1 | B | 100 | 6 | app. 20 |
| CP-3 | P2 | B | 100 | 6 | app. 20 |
| CP-4 | P1/PEG | C | 100 | 6 | 18 |
| CP-5 | P3 | A | 80 | 6 | 20 |
| CP-6 | P3 | A | 150 | 8 | app. 20 |
| CP-7 | P4 | A | 80 | 6 | 8 |
| CP-8 | P4 | A | 160 | 8 | 9 |
| CP-9 | P3/PEG | A | 100 | 6 | 16 |
| CP-10 | P5 | B | 200 | 8 | app. 25 |
| CP-11 | P6 | B | 100 | 6 | 15 |
| CP-12 | P6/PEG | C | 200 | 8 | 17 |
| CP-13 | P7 | A | 100 | 6 | app.20 |
| CP-14 | P7/PEG | A | 100 | 6 | app. 15 |

P1 Polystyrene (PS), 1% divinylbenzene (DVB), 200-400 mesh, Rapp-Polymere (Tuebingen, Germany), cat.-No: H 400 00
P2 PS, 1% divinylbeuzene (DVB), 100-200 mesh Rapp-Polymere, cat.-No: H 200 0
P3 Isocyanato methyl polystyrene (PS-CH₂-NCO), 1% DVB, 200-400 mesh.
Loading (L) = app. 1.0 mmol/g.
Prepared analogously to Booth, R. J.; Hodges, J. C. J. Am. Chem. Soc 1997, 119: 4882-4886, starting from aminomethyl PS (L = app. 1 mmol; Rapp-Polymere, cat.-no: H 400 02)
P4 Isocyanato methyl polystyrene (PS-CH₂-NCO), 1% DVB, 100-200 mesh
L = app. 1.0 mmol/g
Preparation: see under P3
P5 3-(Morpholino)propyl polystyrene sulphonamide
L = app. 2.0 mmol/g, Argonaut (USA); Cat.-No: P/N 800282
P6 (Vinylcarbonyloxymethyl)phenoxymethyl polystyrene (W-COCHCH₂, W = Wang-resin), 1% DVB, 200-400 mesh
L = 0.9 mmol/g, prepared analogous to Morphy, R. J., Rankovic, Rees, D. C. Tetrahedron Lett. 1996, 37: 3209-3212, staffing from Wang-Resin (Rapp Polymere, cat. No: H 2011)
P7 Wang-Resin, 1% DVB, 100-200 mesh, L = 1.0 mmol/g; Rapp-Polymere; cat-No: H 1011
PEG = Dimethylated polyethylene glycol; molecular weight app. 2000 Da; Clariant GmbH (Gendorf, Germany). Material was grinded in a laboratory blender and sieved, particle size varies.
P1/PEG Mixture of P1/PEG = 9:1
P3/PEG Mixture of P3/PEG = 9:1
P6/PEG Mixture of P6/PEG = 9:1
P7/PEG Mixture of P7/PEG = 9:1
A Polymer not pre-treated
B Polymer pre-treated with methylene chloride.
C P1 and P6, rescpectively, pre-treated with methylene chloride, PEG not pre-treated.

### Example 2

Evaluation of Tablets

Disintegration of the Tablets

The tablet was placed in a glass tube (16×100 mm) and treated with 2 mL solvent (see Table 2). The mixture was agitated by vortex mixing at a speed of approximately 500 Hz with an IKA shaker (KS 125 basic). The progress of tablet disintegration was monitored visually. Tablets were deemed to be fully disintegrated when a dispersion was formed in the tube and no more lumbs were present. The results are summarised in Table 2.

TABLE 2
Disintegration in diferent solvents

| | | THF | | | | | |
|---|---|---|---|---|---|---|---|
| | CH₂Cl₂ | in | DMF | Toluene | CH₃CN | DMSO | Ethanol |
| code | in [min] | [min] | in [min] | in [min] | in [min] | in [h] | in [h] |
| CP-1 | <3.0 | <7.0 | <9.0 | <5.0 | >1440* | <2 | >24* |
| CP-2 | <2.5 | <5.0 | <24.0 | <7.0 | >1440* | <12 | <12 |
| CP-3 | <7.0 | <17 0 | <16.0 | <20 0 | >1440* | >24* | >24* |
| CP-4 | <3.0 | <7.0 | <10.0 | <2.0 | <20.0 | <0 333 | <2 |
| CP-5 | <2.5 | <16.0 | <17.0 | <45.0 | >1440* | <0.083 | >24* |
| CP-6 | <2.5 | <3.0 | <3.0 | <5.0 | >1440* | <0.042 | >24* |
| CP-7 | <1.0 | <5.0 | <8.0 | <5.0 | >1440* | <12 | >24* |
| CP-8 | <1.0 | <5.0 | <10.0 | <5.0 | >1440* | <12 | >24* |
| CP-9 | <2.5 | <3.0 | <3.0 | <3.0 | <3.0 | <0.017 | >24* |
| CP-10 | <2.5 | <3.0 | <3.0 | >720 | <5.0 | <2 | >24* |
| CP-11 | <1.0 | <2.0 | <3.0 | <2.0 | <2.0 | <0.083 | >24* |
| CP-12 | <2.5 | <6.0 | <3.0 | <3.0 | <2.0 | <0.183 | >24* |
| CP-13 | <7.0 | <13.0 | <30.0 | <27.0 | <35.0 | <2 | >24* |
| CP-14 | <3.0 | <8.0 | <24.0 | <6 0 | <7.0 | <0 042 | <022 |

*not disintegrated within 1 day

Filterability

After disintegration of the tablet, the filterability of the dispersion was evaluated by using different filter types. All tablets had formed dispersions that readily could be filtered.

### Example 3

Mechanical Stability of the Polymer Beads

A sample of the polymer before tablet formation and a sample of a disintegrated tablet were subjected to SEM analysis using a Philips electron microscope XL30.

The SEM of the polymer before tablet formation shows that the polymer particles are smooth round beads without visible cracks or faults (See FIG. 1).

The SEM of the polymer after disintegration of the tablet shows that the beads are smooth and round without visible deformations and cracks.

This analysis shows that the polymer beads are capable of reshaping after disintegration of the tablet and that no mechanical damage is observed.

### Example 4

Evaluation of the Chemical Performance Before and After Compression of Polymers to Tablets

Comparison of 4-[(4-nitrophenoxy)carbonyloxymethyl]phenoxymethyl Polystyrene as Free Resin and Formulated as a Tablet in the Linking of Amines

4-[(4-Nitrophenoxy)carbonyloxymethyl]phenoxymethyl polystyrene was used for the evaluation of the chemical performance by attachment of different amines and cleavage with TFA/methylene chloride (1:1). Yields and purities of cleaved amines were analysed.

The reactions were performed in a 96-reactor microblock from MultiSynTech. 4-[(4-Nitrophenoxy)carbonyloxymethyl]phenoxymethyl polystyrene (51 mg, 0.42 mmol) was added as tablets to the reactors in the first half (6×8) of the microblock and as free resin to the reactors (6×8) in the second part of the microblock. To each of the eight rows of 12 reactors were added a solution of one of the eight amines selected for the comparison (see below) and N-methyl morpholine (107.0 mg) in DMF (0.7 mL). The reaction mixtures were agitated by shaking at room temperature for 16 h. The resin was filtered and washed with DMF (2×1 mL), MeOH (1×1 mL), THF (1×1 mL), MeOH (1×1 mL), THF (1×1 mL), MeOH (1×1 mL), and methylene chloride (5×1 mL). The resin was treated with a solution of methylene chloride/trifluoro-acetic acid (1:1) (0.65 mL) at room temperature for 2 h, then filtered and washed with methylene chloride (1×1 mL), MeOH (1×1 mL), and methylene chloride (1×1 mL). The filtrates were combined and the volatile solvents evaporated. The residue was dissolved in MeOH (2 mL) and purified using SCX. After evaporation of the solvents, the remaining residue was weighed and re-desolved in DMSO and analysed by LC/MS using ELSD and UV detection. Yields and purities reported in table 4 are average values of the six simultaneous reactions in a row of reactors.

The following amines have been used according to the procedure described: 1-(2-Piperazin-1-yl-ethyl)-imidazolidin-2-one (entry 1); 4-(10,10-dimethyl-4a,9,9a, 10-tetrahydro-anthracen-9-yl)-piperidine (entry 2); 1-benzyl-piperidin-4-ylamine (entry 3); 2-(3,4-dimethoxy-phenyl)-ethylamine (entry 4); dibenzyl-amine (entry 5); 3,3-diphenyl-propylamine (entry 6); benzyl-cyclopropyl-amine (entry 7); 8-fluoro-2,3,4,5,5a,9a-hexahydro-1H-pyrido[4,3-b]indole (entry 8).

TABLE 4
Results from example 4

| | Starting amine | | | Free resin | | | Tablet formulated resin | | |
|---|---|---|---|---|---|---|---|---|---|
| | | Purity | Purity | | Purity | Purity | | Purity | Purity |
| Entry | Amine | UV (%) | ELSD (%) | Yield (%) | UV (%) | ELSD (%) | Yield (%) | UV (%) | ELSD (%) |
| 1 | 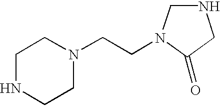 | — | 94 | 70 | — | 100 | 105 | — | 100 |
| 2 | 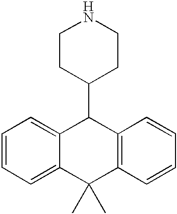 | 94 | 100 | 65 | 86 | 87 | 105 | 95 | 97 |
| 3 | 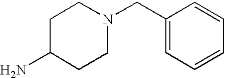 | 90 | 92 | 62 | 70 | 100 | 90 | 75 | 100 |
| 4 | 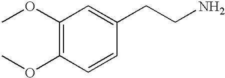 | 85 | 100 | 59 | 44 | 49 | 86 | 61 | 70 |
| 5 | 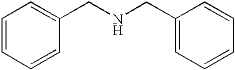 | 54 | 100 | 50 | 80 | 98 | 83 | 91 | 100 |
| 6 | 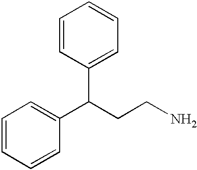 | 78 | 99 | 62 | 83 | 94 | 98 | 89 | 99 |
| 7 | 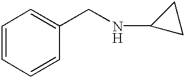 | 45 | 98 | 19 | 31 | 70 | 9 | 24 | 50 |
| 8 | 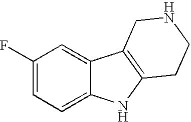 | 98 | 99 | 72 | 56 | 56 | 89 | 67 | 70 |

What is claimed is:

1. A dosing form for a polymer support for organic chemical synthesis in a solvent medium comprising compressed tablets of essentially equal weight and composition, each comprising essentially the same weight amount of beads of a polymer containing functional groups, which polymer is insoluble in the solvent for the intended synthesis, and said tablets being capable of disintegrating in said solvent thereby releasing the polymer beads essentially intact, wherein said tablets comprise less than 20 percent by weight of polyethylene glycol.

2. The dosing form according to claim 1, wherein the tablets are capable of disintegration in the intended solvent within 10 minutes.

3. The dosing form according to claim 1, wherein the polymer is insoluble in an organic solvent.

4. The dosing form according to claim 1, wherein the tablets further comprise a polymer without functional groups.

5. The dosing form according to claim 1, wherein the polymer containing functional groups is selected among: polystyrene, linear polystyrene, polystyrene crosslinked with divinylbenzene, polystyrene crosslinked with polyethylene glycol including the POEPS and POEPS-3 resins, polystyrene resins cross-linked with polyoxybutylene, polyethylene glycol grafted resins, polyoxyethylene polyoxy propylene resins and polymers co-polymerized together with magnetites or magnetites captured in highly crosslinked polystyrene particles.

6. The dosing form according to claim 5, wherein the polymer containing functional groups are based on polystyrene.

7. The dosing form according to claim 1, wherein the polymer containing functional groups is selected from the group comprising TentaGel® resins (Rapp polymere GmbH, Tübingen, Germany), ArgoGel® resins (Sigma-Aldrich), Merrifield-resin and Novasyn® resins (Calbiochem-Novabiochem AG Schwizerland), in particular: TentaGel S AC, TentaGel S Trt, TentaGel S PHB, TentaGel S HMB, TentaGel S AM, TentaGel S RAM, ArgoGel™-AS-SO₂NH₂, ArgoGel™-Cl, ArgoGel™-MB-CHO, ArgoGel™-MB-OH, ArgoGel™-NH₂, ArgoGel™-OH, ArgoGel™-Rink, ArgoGel™-Wang, ArgoGel™-Wang-Cl, aminomethylated poly(styrene-co-divinylbenzene), polymer bound piperidine, polymer bound 4-benzyloxybenzaldehyde, polymer bound isocyanate, polymer bound diethylenetriamine, vinylsulfonylmethyl polystyrene, acryloyl Wang resin, chloromethylpolystyrene-divinylbenzene, brominated Wang resin, 2-chlorotrityl chloride resin, brominated PPOA resin, NovaSyn® TGT alcohol resin, trityl chloride resin, 4-methyltrityl chloride resin, 4-methoxytrityl chloride resin, NovaSyn® trityl alcohol resin, NovaSyn® TG bromo resin, NovaSyn® dichlorotrityl alcohol TG resin, bromo-(2-chlorophenyl)methyl polystyrene, bromo-(4-methoxyphenyl)methyl polystyrene, 4-bromo polystyrene, 4-(bromomethyl) phenoxyethyl polystyrene, bromoacetamidomethyl NovaGel™, bromomethylphenyl-acetamidomethyl NovaGel™, p-nitrophenyl carbonate Wang resin, p-nitrophenyl carbonate Merrifield resin, formylpolystyrene, NovaSyn® TG acetal resin, 2-(4-formyl-3-methoxyphenoxy)ethyl polystyrene, 2-(3,5-dimethoxy-4-formylphenoxy)ethoxymethyl polystyrene, 4-(4-formyl-3-methoxyphenoxy)butyryl NovaGel™ HL, carboxypolystyrene HL, 4-methylbenzhydrylamine resin LL, aminomethylated polystyrene HL, 4-(2′,4′-dimethoxyphenyl-FMOC-aminomethyl)-phenoxyacetamido-norleucin-MBHA resin, 4-methylbenzhydrylamine resin HL, hydrazine 2-chlorotrityl resin, 9-FMOC-amino-xanthen-3-yloxy-methyl resin, NovaSyn® TG amino resin HL, 4-sulfamylbutyryl AM resin, 4-sulfamylbenzoyl AM resin, 4-sulfamylbenzoyl NovaSyn® TG resin, amino-(4-methoxyphenyl)methyl polystyrene, aminomethyl NovaGel™, Rink amide NovaGel™, 4-sulfamylbenzoyl NovaGel™, 4-FMOC-hydrazinobenzoyl AM resin, N-benzylaminomethyl polystyrene, piperazinomethyl polystyrene, N-methylaminomethyl polystyrene, Wang resin, Rink acid resin, oxime resin LL, HMPB-BHA resin, 4-Hydroxymethylphenoxyacetyl PEGA resin, hydroxymethyl polystyrene, 4-hydroxymethylbenzoic acid PEGA resin, 4-hydroxymethylbenzoic acid AM resin, 4-(2′,4′-dimethoxyphenyl-hydroxymethyl)-phenoxy resin, methylisocyanate polystyrene HL, tris-(2-aminoethyl)-amine polystyrene HL, morpholinomethyl polystyrene HL, N-(2-aminoethyl)aminomethyl polystyrene, bis-HOBt-ethylenediamine methyl polystyrene, cysteamine methyl polystyrene, thiocarbamoyl AM resin, N-cyclohexylcarbodiimide, N+-methyl polystyrene HL, 4-(N-benzyl-N-methylamino)-pyridine polymer supported, ethylene glycol polymer bound, 4-maleimidobutyramidomethyl-polystyrene, polyethylene glycol 600 bound to polystyrene-1% DVB, poly(4-vinylpyridine), poly(4-vinylpyridinium hydrochloride), poly(4-vinylpyridinium dichromate), poly[4-vinylpyridinium poly(hydrogen flouride)], poly(4-vinylpyridinium p-toluenesulphonate), poly(4-vinylpyridinium tribromide), pyridinium chlorochromate polymer bound, pyridinium toluene-4-sulfonate polymer bound, sulfur trioxide pyridine complex polymer bound, thiocyanate polymer supported, 1,5,7-triazabicyclo[4.4.0]dec-5-ene on polystyrene, tributylmethylammonium chloride polymer bound, tributylmethylphosphonium chloride polymer bound and triphenylphosphine polymer bound.

8. The dosing form according to claim 1, wherein the tablets further comprise an additive.

9. The dosing form according to claim 8, wherein the additive comprises an disintegrating agent.

10. The dosing form according to claim 9, wherein the disintegrating agent is polystyrene or dimethylated polyethylene glycol having a molecular weight of about 2000 Da (DM-PEG 2000) or higher.

11. The dosing form according to claim 1, wherein the tablets are uncoated.

12. A method of carrying out a parallel synthesis, split and mix synthesis or combinatorial chemistry with a dosing form according to claim 1.

13. A method for producing a dosing form for a polymer support for organic chemical synthesis in a solvent medium comprising compression of beads of a polymer containing functional groups, which polymer is insoluble in the solvent for the intended synthesis into tablets of essentially equal weight and composition, each comprising essentially the same weight amount of said polymer and said tablets being capable of disintegrating in said solvent thereby releasing the polymer beads essentially intact, wherein the polymer having functional groups or a mixture comprising said polymer is pre-treated with an aprotic organic solvent and dried before tablet formation.

14. The method according to claim 13, wherein the pre-treatment comprises suspending the polymer having functional groups or the mixture comprising said polymer in an aprotic organic solvent and subsequently drying said polymer or mixture before tablet formation.

15. The method according to claim 14, wherein the solvent is methylene chloride or tetrahydrofuran.

16. The method according to claim 13, wherein the material used for tabletting comprises less than 20 percent by weight of polyethylene glycol.

17. The method according to claim 13, wherein the tablets produced are capable of disintegration in the intended solvent within 10 minutes.

18. The method according to claim 13, wherein the polymer is insoluble in an organic solvent.

19. The method according to claim 13, wherein the tablets further comprise a polymer without functional groups.

20. The method according to claim 13, wherein the polymer containing functional groups is selected among: polystyrene, linear polystyrene, polystyrene cross-linked with divinylbenzene, polystyrene cross-linked with polyethylene glycol including the POEPS and POEPS-3 resins, polystyrene resins crosslinked with polyoxybutylene, polyethylene glycol grafted resins, polyoxyethylene polyoxy propylene resins and polymers co-polymerized together with magnetites or magnetites captured in highly crosslinked polystyrene particles.

21. The method according to claim 20, wherein the polymer containing functional groups are based on polystyrene.

22. The method according to claim 13, wherein the polymer containing functional groups is selected from the group comprising TentaGel® resins (Rapp polymere GmbH, Tübingen, Germany), ArgoGel® resins (Sigma-Aldrich), Merrifield-resin and Novasyn® resins (Calbiochem-Novabiochem AG Schwizerland), in particular: TentaGel S AC, TentaGel S Trt, TentaGel S PHB, TentaGel S HMB, TentaGel S AM, TentaGel S RAM, ArgoGel™-AS-SO₂NH₂, ArgoGel™-Cl, ArgoGel™-MB-CHO, ArgoGel™-MB-OH, ArgoGel™-NH₂, ArgoGel™-OH, ArgoGel™-Rink, ArgoGel™-Wang, ArgoGel™-Wang-Cl, aminomethylated poly(styrene-co-divinylbenzene), polymer bound piperidine, polymer bound 4-benzyloxybenzaldehyde, polymer bound isocyanate, polymer bound diethylenetriamine, vinylsulfonylmethyl polystyrene, acryloyl Wang resin, chloromethylpolystyrene-divinylbenzene, brominated Wang resin, 2-chlorotrityl chloride resin, brominated PPOA resin, NovaSyn® TGT alcohol resin, trityl chloride resin, 4-methyltrityl chloride resin, 4-methoxytrityl chloride resin, NovaSyn® trityl alcohol resin, NovaSyn® TG bromo resin, NovaSyn® dichlorotrityl alcohol TG resin, bromo-(2-chlorophenyl)methyl polystyrene, bromo-(4-methoxyphenyl)methyl polystyrene, 4-bromo polystyrene, 4-(bromomethyl) phenoxyethyl polystyrene, bromoacetamidomethyl NovaGel™ bromomethylphenyl-acetamidomethyl NovaGel™, p-nitrophenyl carbonate Wang resin, p-nitrophenyl carbonate Merrifield resin, formylpolystyrene, NovaSyn® TG acetal resin, 2-(4-formyl-3-methoxyphenoxy)ethyl polystyrene, 2-(3,5-dimethoxy-4-formylphenoxy)ethoxymethyl polystyrene, 4-(4-formyl-3-methoxyphenoxy)butyryl NovaGel™ HL, carboxypolystyrene HL, 4-methylbenzhydrylamine resin LL, aminomethylated polystyrene HL, 4-(2′,4′-dimethoxyphenyl-FMOC-aminomethyl)-phenoxyacetamido-norleucin-MBHA resin, 4-methylbenzhydrylamine resin HL, hydrazine 2-chlorotrityl resin, 9-FMOC-amino-xanthen-3-yloxy-methyl resin, NovaSyn® TG amino resin HL, 4-sulfamylbutyryl AM resin, 4-sulfamylbenzoyl AM resin, 4-sulfamylbenzoyl NovaSyn® TG resin, amino-(4-methoxyphenyl)methyl polystyrene, aminomethyl NovaGel™, Rink amide NovaGel™, 4-sulfamylbenzoyl NovaGel™, 4-FMOC-hydrazinobenzoyl AM resin, N-benzylaminomethyl polystyrene, piperazinomethyl polystyrene, N-methylaminomethyl polystyrene, Wang resin, Rink acid resin, oxime resin LL, HMPB-BHA resin, 4-Hydroxymethylphenoxyacetyl PEGA resin, hydroxymethyl polystyrene, 4-hydroxymethylbenzoic acid PEGA resin, 4-hydroxymethylbenzoic acid AM resin, 4-(2′,4′-dimethoxyphenyl-hydroxymethyl)-phenoxy resin, methylisocyanate polystyrene HL, tris-(2-aminoethyl)-amine polystyrene HL, morpholinomethyl polystyrene HL, N-(2-aminoethyl)aminomethyl polystyrene, bis-HOBt-ethylenediamine methyl polystyrene, cysteamine methyl polystyrene, thiocarbamoyl AM resin, N-cyclohexylcarbodiimide, N+-methyl polystyrene HL, 4-(N-benzyl-N-methylamino)-pyridine polymer supported, ethylene glycol polymer bound, 4-maleimidobutyramidomethyl-polystyrene, polyethylene glycol 600 bound to polystyrene-1% DVB, poly(4-vinylpyridine), poly(4-vinylpyridinium hydrochloride), poly(4-vinylpyridinium dichromate), poly[4-vinylpyridinium poly(hydrogen flouride)], poly(4-vinylpyridinium p-toluenesulphonate), poly(4-vinylpyridinium tribromide), pyridinium chlorochromate polymer bound, pyridinium toluene-4-sulfonate polymer bound, sulfur trioxide pyridine complex polymer bound, thiocyanate polymer supported, 1,5,7-triazabicyclo[4.4.0]dec-5-ene on polystyrene, tributylmethylammonium chloride polymer bound, tributylmethylphosphonium chloride polymer bound and triphenylphosphine polymer bound.

23. The method according to claim 13, wherein the material used for tabletting further comprises an additive.

24. The method according to claim 23, wherein the additive comprises an disintegrating agent.

25. The method according to claim 24, wherein the disintegrating agent is polystyrene or dimethylated polyethylene glycol having a molecular weight of about 2000 Da (DM-PEG 2000) or higher.

26. A dosing form for a polymer support for organic chemical synthesis in a solvent medium comprising compressed tablets of essentially equal weight and composition each comprising essentially the same weight amount of beads of a polymer containing functional groups which polymer is insoluble in the solvent for the intended synthesis and said tablets being capable of disintegrating in said solvent thereby releasing the polymer beads essentially intact, wherein said tablets are made by a method according to claim 13.

27. A method of carrying out a parallel synthesis, split and mix synthesis or combinatorial chemistry using a dosing form according to claim 26.
